(19) United States
(12) Patent Application Publication
(10) Pub. No.: US 2022/0288171 A1
(43) Pub. Date: Sep. 15, 2022

(54) TROPOELASTIN FOR USE IN TREATMENT OF ACNE SCARRING

(71) Applicant: ALLERGAN PHARMACEUTICALS INTERNATIONAL LIMITED, Dublin (IE)

(72) Inventors: Robert DANIELS, Sydney (AU); Caroline COLLINS, Dublin (IE); John St. Clair ROBERTS, Suffolk (GB); John WESTWATER, Dublin (IE)

(21) Appl. No.: 17/637,799

(22) PCT Filed: Aug. 21, 2020
(86) PCT No.: PCT/EP2020/073516

§ 371 Date: Feb. 23, 2022

Related U.S. Application Data
(60) Provisional application No. 62/891,232, filed on Aug. 23, 2019.

(51) Int. Cl. A61K 38/39; A61K 31/728; A61K 9/00; A61P 17/02

(52) U.S. Cl. CPC A61K 38/39; A61K 31/728; A61K 9/0014; A61P 17/02

(57) ABSTRACT

The present disclosure provides methods for the treatment of an acne scar to improve color and/or appearance, and/or reduce depth, affected area, and/or volume of the acne scar. Such methods may comprise comprise administering a composition that comprises tropoelastin to an area of skin having the acne scar and optionally disrupting the fibrotic strands underneath the acne scar and administering the composition underneath the acne scar.

### FIELD

This disclosure relates to methods of reducing the appearance of acne scars, such as depressions, ice pick scars, rolling atrophic acne scars and boxcar scars using a composition that comprises tropoelastin.

### BACKGROUND

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Acne scars may be a result of infected blemishes caused by clogged skin pores that are engorged with excess oil, dead skin cells and bacteria. The pore swells, causing a break in the follicle wall. Shallow lesions are usually minor and may heal quickly. But if there is a deep break in the wall of the pore, infected material can spill out into surrounding tissue, creating a deeper lesion. Thus, the skin may attempt to repair these lesions by forming new collagen fibers. In some cases, a visible scar can be in a prominent location, such as the face, neck and chest which may be an annoyance to a person. There are multiple variables that may affect the severity of the scarring, such as the thickness and coloring of the scars. These repairs may not be as smooth and flawless as the original skin. Additionally, healing may lead to unwanted coloring of the skin within the scar.

There are several types of acne scars. Without being limiting, there are hypertrophic, ice pick scarring, boxcar type scarring, rolling atrophic scarring and depressed scarring. In some cases, the scarring can lead to discoloration of the skin, exposure to UV can cause the scars to darken and increase their prominence, and fibrous tissue in the scar may be silvery in color.

There are several types of treatments that have been described to prevent and to treat acne, however, it is the scarring that is left behind which may be difficult to manage and tougher to control. For example, common treatments for the skin include using topical retinoids to fade brown, red or purple discoloration left behind by acne, laser treatments, dermabrasion and punch grafting. As such, there is a need to treat unsightly scarring and improve its appearance. There is also a desire to reduce the depth, affected area and volume of depressions that may be caused by acne.

### SUMMARY

The present disclosure generally relates to compositions that comprise tropoelastin and methods of using such compositions for the treatment of acne scars (e.g., fibrotic acne scars). The methods may comprise administering a composition that comprises tropoelastin to an area of skin having an acne scar. In an embodiment, the methods comprise treating an area of skin having an acne scar to improve color and/or appearance, and/or reduce depth, affected area, and/or volume of the acne scar in an area of skin of a patient in need thereof. The treatment methods disclosed herein may improve color and/or appearance of the acne scar in the area of skin of a patient in need thereof. Additionally or alternatively, the treatment method may reduce depth, affected area and/or volume of the acne scar in the area of skin of a patient in need thereof

In some embodiments of each or any of the above- or below-mentioned embodiments, the composition comprises between about 1 mg/ml to about 400 mg/ml tropoelastin. In some embodiments of each or any of the above- or below-mentioned embodiments, the composition comprises about 1 mg/ml, about 5 mg/ml, about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 210 mg/ml, about 220 mg/ml, about 230 mg/ml, about 240 mg/ml, about 250 mg/ml, about 260 mg/ml, about 270 mg/ml, about 280 mg/ml, about 290 mg/ml, about 300 mg/ml, about 310 mg/ml, about 320 mg/ml, about 330 mg/ml, about 340 mg/ml, about 350 mg/ml, about 360 mg/ml, about 370 mg/ml, about 380 mg/ml, about 390 mg/ml or about 400 mg/ml tropoelastin or any amount of tropoelastin in between a range defined by any two aforementioned values.

In some embodiments of each or any of the above- or below-mentioned embodiments, the composition comprises between about 1 mg/ml to about 300 mg/ml tropoelastin. In some embodiments of each or any of the above- or below-mentioned embodiments, the composition comprises between about 1 mg/ml to about 250 mg/ml tropoelastin. In some embodiments of each or any of the above- or below-mentioned embodiments, the composition comprises between about 1 mg/ml to about 200 mg/ml tropoelastin. In some embodiments of each or any of the above- or below-mentioned embodiments, the composition comprises between about 1 mg/ml to about 150 mg/ml tropoelastin. In some embodiments of each or any of the above- or below-mentioned embodiments, the composition comprises between about 1 mg/ml to about 100 mg/ml tropoelastin.

In some embodiments of each or any of the above- or below-mentioned embodiments, the tropoelastin is crosslinked with about 0.1% to about 10% derivatized hyaluronic acid. In some embodiments of each or any of the above- or below-mentioned embodiments, the composition comprises between about 1 mg/ml to about 100 mg/ml tropoelastin crosslinked with about 0.4% to about 1% derivatized hyaluronic acid (HA). In some embodiments of each or any of the above- or below-mentioned embodiments, the composition comprises about 30 mg/ml recombinant human tropoelastin crosslinked with about 0.5% derivatized hyaluronic acid. In some embodiments of each or any of the above- or below-mentioned embodiments, the composition further comprises a buffer (e.g., phosphate buffered saline).

In some embodiments of each or any of the above- or below-mentioned embodiments, the composition comprising tropoelastin is administered around the scar, such as beneath the scar and/or around edges of the scar.

In some embodiments of each or any of the above- or below-mentioned embodiments, the acne scars are sub-classified as ice pick scars, boxcar scars or rolling atrophic scars. In some embodiments of each or any of the above- or below-mentioned embodiments, the acne scar is an ice pick scar. In some embodiments of each or any of the above- or below-mentioned embodiments, the acne scar is a box scar. In some embodiments of each or any of the above- or below-mentioned embodiments, the scar is a rolling atrophic acne scar. In some embodiments of each or any of the above- or below-mentioned embodiments, the scar is a hypertrophic scar. In some embodiments of each or any of the above- or below-mentioned embodiments, the scar comprises fibrotic tissue around the edges or bottom of the scar.

In some embodiments of each or any of the above- or below-mentioned embodiments, the acne scar has a depth of about 0.1 mm to about 5 mm. In some embodiments of each or any of the above- or below-mentioned embodiments, the acne scar has a depth of about 0.1 mm, about 0.5 mm, about 1.0 mm, about 1.5 mm, about 2 mm, about 2.5 mm, about 3.0 mm, about 3.5 mm, about 4.0 mm, about 4.5 mm, about 5 mm, or any depth in a range in between any two aforementioned values.

In some embodiments of each or any of the above- or below-mentioned embodiments, the acne scar has an affected area of about 0.05 mm² to about 400 mm². In some embodiments of each or any of the above- or below-mentioned embodiments, the acne scar has an affected area of about 0.05 mm², about 0.50 mm², 1.0 mm², about 5 mm², about 10 mm², about 15 mm², about 20 mm², about 25 mm², about 30 mm², about 35 mm², about 40 mm², about 45 mm², about 50 mm², about 55 mm², about 60 mm², about 65 mm², about 70 mm², about 75 mm², about 80 mm², about 85 mm², about 90 mm², about 95 mm², about 100 mm², about 125 mm², about 150 mm², about 175 mm², about 200 mm², about 225 mm², about 250 mm², about 275 mm², about 300 mm², about 325 mm², about 350 mm², about 375 mm², or about 400 mm² or any value in a range in between any two aforementioned values.

In some embodiments of each or any of the above- or below-mentioned embodiments, the acne scar has a volume of about 0.01 mm³ to about 2,000 mm³. In some embodiments of each or any of the above- or below-mentioned embodiments, the acne scar has a volume of about 0.01 mm³, 1 mm³, about 10 mm³, about 20 mm³, about 30 mm³, about 40 mm³, about 50 mm³, about 60 mm³, about 70 mm³, about 80 mm³, about 90 mm³, about 100 mm³, about 125 mm³, about 150 mm³, about 175 mm³, about 200 mm³, about 225 mm³, about 250 mm³, about 275 mm³, about 300 mm³, about 325 mm³, about 350 mm³, about 375 mm³, about 400 mm³, about 425 mm³, about 450 mm³, about 475 mm³, about 500 mm³, about 525 mm³, about 550 mm³, about 575 mm³, about 600 mm³, about 625 mm³, about 650 mm³, about 675 mm³, about 700 mm³, about 725 mm³, about 750 mm³, about 775 mm³, about 800 mm³, about 825 mm³, about 850 mm³, about 875 mm³, about 900 mm³, about 925 mm³, about 950 mm³, 975 mm3, about 1,000 mm³, about 1,100 mm³, about 1,200 mm³, about 1,300 mm³, about 1,400 mm³, about 1,500 mm³, about 1,600 mm³, about 1,700 mm³, about 1,800 mm³, about 1,900 mm³, or about 2,000 mm³, or any volume in a range between any two aforementioned values.

In some embodiments of each or any of the above- or below-mentioned embodiments, the method further comprises a step of disrupting fibrotic strands underneath the acne scar. In some embodiments of each or any of the above- or below-mentioned embodiments, the disrupting is performed prior to administration of the composition to the patient in need. In some embodiments of each or any of the above- or below-mentioned embodiments, the step of disrupting the fibrotic strands creates a dermal pocket underneath the acne scar. In some embodiments of each or any of the above- or below-mentioned embodiments, the disrupting is performed with a 18-32G needle, such as an 18G, 21G, 23G, 25G, 27G, 29G or 30G needle. In some embodiments of each or any of the above- or below-mentioned embodiments, the administering comprises injecting the composition into the dermal pocket. In some embodiments of each or any of the above- or below-mentioned embodiments, the composition comprising tropoelastin is also placed around the scar, such as beneath the scar and around the edges of the scar. An even placement of the composition comprising tropoelastin around the scar throughout the dermis surrounding the scar may ensure that the tropoelastin product is around the scar area.

In some embodiments of each or any of the above- or below-mentioned embodiments, the composition is administered as an injection beneath the acne scar. In some embodiments of each or any of the above- or below-mentioned embodiments, the composition is administered in a volume of about 10 μL to about 100 μL per implant/injection. In some embodiments of each or any of the above- or below-mentioned embodiments, the composition is administered in a volume of about 10 μL, about 20 μL, about 30 μL, about 40 μL, about 50 μL, about 60 μL, about 70 μL, about 80 μL, about 90 μL, or about 100 μL or within a range defined by any two aforementioned values. In some embodiments, more than one injection is made into the acne scar and the total administered volume is about 200 μL, about 300 μL, about 400 μL, or about 500 μL.

In some embodiments of each or any of the above- or below-mentioned embodiments, the injection is given using a retrograde linear threading technique in a cross-hatching arrangement to ensure any fibrous strands within the acne scar are disrupted. In some embodiments of each or any of the above- or below-mentioned embodiments, the needle is inserted at an angle of about 30° parallel to the skin, wherein the needle comprises a bevel and the bevel is facing upwards. In some embodiments of each or any of the above- or below-mentioned embodiments, the needle is inserted more than one time to break up the fibrous strands and create the dermal pocket. In some embodiments of each or any of the above- or below-mentioned embodiments, even pressure is applied to inject the composition at the same time as the needle is withdrawn from the dermal pocket.

In some embodiments of each or any of the above- or below-mentioned embodiments, the administering of the composition is repeated, wherein one or more bolus injections into the scar is administered. In some embodiments of each or any of the above- or below-mentioned embodiments, a maximum volume of composition given as a treatment is between about 100 μL to about 5 mL. In some embodiments of each or any of the above- or below-mentioned embodiments, a maximum volume of composition given as a treatment is about 100 μL , about 500 μL , about 1 ml, about 1.5 ml, about 2 ml, about 2.5 ml, about 3 ml, about 3.5 ml, about 4.0 ml, about 4.5 ml, about 5 ml or any amount in between a range defined by any two aforementioned values. In some embodiments of each or any of the above- or below-mentioned embodiments, the maximum volume of composition given as a treatment is between about 100 μL to about 500 μL per square cm. In some embodiments of each or any of the above- or below-mentioned embodiments, the maximum volume of composition given as a treatment is about 100 μL per square cm, about 150 μL per square cm, about 200 μL per square cm, about 250 μL per square cm, about 300 μL per square cm, about 350 μL per square cm, about 400 μL per square cm, about 450 μL per square cm, about 500 μL per square cm or any amount in between a range defined by any two aforementioned values.

In some embodiments of each or any of the above- or below-mentioned embodiments, the patient in need has a skin type on a Fitzpatrick skin type scale of I, II, III, IV, V or VI. In some embodiments of each or any of the above- or below-mentioned embodiments, the scar comprises a grade of 1-5. The scar grade is as described in a Global Scale for Acne Scar Severity (SCAR-S) by Tan et al. 2010 (Journal of Cutaneous Medicine and Surgery, Vol 14, No 4 (July/August), incorporated by reference herein).

In some embodiments of each or any of the above- or below-mentioned embodiments, the acne scars are subclassified as ice pick scars, boxcar scars or rolling atrophic scars. In some embodiments of each or any of the above- or below-mentioned embodiments, the acne scar is an ice pick scar. In some embodiments of each or any of the above- or below-mentioned embodiments, the acne scar is a box scar. In some embodiments of each or any of the above- or below-mentioned embodiments, the scar is a rolling atrophic acne scar. In some embodiments of each or any of the above- or below-mentioned embodiments, the scar is a hypertrophic scar. In some embodiments of each or any of the above- or below-mentioned embodiments, the acne scar comprises fibrotic tissue around the edges of the scar and/or bottom of the scar.

In some embodiments of each or any of the above- or below-mentioned embodiments, the acne scar has a depth of about 0.1 mm to about 5 mm. In some embodiments of each or any of the above- or below-mentioned embodiments, the acne scar has a depth of about 0.1 mm, about 0.5 mm, about 1.0 mm, about 1.5 mm, about 2 mm, about 2.5 mm, about 3.0 mm, about 3.5 mm. about 4.0 mm, about 4.5 mm, about 5 mm, or any depth in a range in between any two aforementioned values. In some embodiments of each or any of the above- or below-mentioned embodiments, the acne scar has an affected area of about 0.05 mm² to about 400 mm². In some embodiments of each or any of the above- or below-mentioned embodiments, the acne scar has a volume of about 0.01 mm³ to 2,000 mm³.

In some embodiments of each or any of the above- or below-mentioned embodiments, the acne scar has a volume of about 0.01 mm³, 1 mm³, about 10 mm³, about 20 mm³, about 30 mm³, about 40 mm³, about 50 mm³, about 60 mm³, about 70 mm³, about 80 mm³, about 90 mm³, about 100 mm³, about 125 mm³, about 150 mm³, about 175 mm³, about 200 mm³, about 225 mm³, about 250 mm³, about 275 mm³, about 300 mm³, about 325 mm³, about 350 mm³, about 375 mm³, about 400 mm³, about 425 mm³, about 450 mm³, about 475 mm³, about 500 mm³, about 525 mm³, about 550 mm³, about 575 mm³, about 600 mm³, about 625 mm³, about 650 mm³, about 675 mm³, about 700 mm³, about 725 mm³, about 750 mm³, about 775 mm³, about 800 mm³, about 825 mm³, about 850 mm³, about 875 mm³, about 900 mm³, about 925 mm³, about 950 mm³, 975 mm3, about 1,000 mm³, about 1,100 mm³, about 1,200 mm³, about 1,300 mm³, about 1,400 mm³, about 1,500 mm³, about 1,600 mm³, about 1,700 mm³, about 1,800 mm³, about 1,900 mm³, or about 2,000 mm³, or any volume in a range between any two aforementioned values.

In some embodiments of each or any of the above- or below-mentioned embodiments, the skin comprises a skin color defined by CIE L*a*b* color coordinates, wherein the method further decreases L*. In some embodiments of each or any of the above- or below-mentioned embodiments, the method decreases L* by about 1%, about 5%, about 10%, about 15%, about 20%, about 25% or greater than about 50%, or any amount defined by a range in between any two aforementioned values. In some embodiments of each or any of the above- or below-mentioned embodiments, the method decreases L* greater than about 50%. In some embodiments of each or any of the above- or below-mentioned embodiments, the method further increases a*. In some embodiments of each or any of the above- or below-mentioned embodiments, the method increases a* by about 1%, about 5%, about 10%, about 15%, about 20%, about 25% or greater than about 50%, or any amount defined by a range in between any two aforementioned values. In some embodiments of each or any of the above- or below-mentioned embodiments, the method increases a* by greater than about 50%. In some embodiments of each or any of the above- or below-mentioned embodiments, the scar comprises a silver coloring prior to administering the composition. In some embodiments of each or any of the above- or below-mentioned embodiments, administering the composition decreases the silver coloring and increases red coloring and/or pink coloring of the scar. In some embodiments of any one of each or any of the above- or below-mentioned embodiments, the method decreases L* by about 1%, about 5%, about 10%, about 15%, about 20%, about 25% or greater than about 50%, or any amount defined by a range in between any two aforementioned values and increases a* by about 1%, about 5%, about 10%, about 15%, about 20%, about 25% or greater than about 50%, or any amount defined by a range in between any two aforementioned values. In some embodiments of each or any of the above- or below-mentioned embodiments, the scar comprises a color, wherein the color is different from a natural skin coloring of the patient, wherein administering the composition results in the color of the scar decreasing or fading in color intensity such that the color of the scar integrates into the natural skin coloring of the patient.

In some embodiments of each or any of the above- or below-mentioned embodiments, the acne scar is a facial, back, or a torso scar. In some embodiments of each or any of the above- or below-mentioned embodiments, the depth, affected area and/or volume of the acne scar is reduced following administering of the composition.

In some embodiments of each or any of the above- or below-mentioned embodiments, the method supports the repair of an atrophic scar during the skin remodeling and maturation phase to reduce the appearance of the acne scar.

In some embodiments of each or any of the above- or below-mentioned embodiments, the method provides a reduction in the acne scar area by about 10%, about 20%, about 30%, about 40%, about 50%, about 60%, about 70%, about 80% or greater than about 90% or any amount in between a range described by any two aforementioned values. In some embodiments of each or any of the above- or below-mentioned embodiments, the method provides a reduction in the acne scar area by greater than about 90%. In some embodiments of each or any of the above- or below-mentioned embodiments, the composition provides a reduction in the acne scar area by about 10%, about 20%, about 30%, about 40%, about 50%, about 60%, about 70%, about 80% or greater than about 90% or any amount in between a range described by any two aforementioned values. In some embodiments of each or any of the above- or below-mentioned embodiments, the composition provides a reduction in the acne scar area by greater than about 90%.

In some embodiments of each or any of the above- or below-mentioned embodiments, the method provides a reduction in the acne scar volume by about 10%, about 20%, about 30%, about 40%, about 50%, about 60%, about 70%, about 80% or greater than about 90% or any amount in between a range described by any two aforementioned values. In some embodiments of each or any of the above- or below-mentioned embodiments, the method provides a reduction in the acne scar volume by greater than about 90%. In some embodiments of each or any of the above- or below-mentioned embodiments, the composition provides a reduction in the acne scar volume by about 10%, about 20%, about 30%, about 40%, about 50%, about 60%, about 70%, about 80% or greater than about 90% or any amount in between a range described by any two aforementioned values. In some embodiments of each or any of the above- or below-mentioned embodiments, the composition provides a reduction in the acne scar volume by greater than about 90%.

In some embodiments of each or any of the above- or below-mentioned embodiments, the method provides a reduction in the acne scar depth by about 10%, about 20%, about 30%, about 40%, about 50%, about 60%, about 70%, about 80% or greater than about 90% or any amount in between a range described by any two aforementioned values. In some embodiments of each or any of the above- or below-mentioned embodiments, the method provides a reduction in the acne scar depth by greater than about 90%. In some embodiments of each or any of the above- or below-mentioned embodiments, the composition provides a reduction in the acne scar depth by about 10%, about 20%, about 30%, about 40%, about 50%, about 60%, about 70%, about 80% or greater than about 90% or any amount in between a range described by any two aforementioned values. In some embodiments of each or any of the above- or below-mentioned embodiments, the composition provides a reduction in the acne scar depth by greater than about 90%.

In some embodiments of each or any of the above- or below-mentioned embodiments, wherein the patient in need has a region of acne scarring on the skin, the method provides a reduction in the depression volume and area of skin contours in the region of acne scarring by about 10%, about 20%, about 30%, about 40%, about 50%, about 60%, about 70%, about 80% or greater than about 90% or any amount in between a range described by any two aforementioned values. In some embodiments of each or any of the above- or below-mentioned embodiments, wherein the patient in need has a region of acne scarring on the skin, the method provides a reduction in the depression volume and area of skin contours in the region of acne scarring by greater than about 90%. In some embodiments of each or any of the above- or below-mentioned embodiments, the composition provides a reduction in the depression volume and area of skin contours in the region of acne scarring by about 10%, about 20%, about 30%, about 40%, about 50%, about 60%, about 70%, about 80% or greater than about 90% or any amount in between a range described by any two aforementioned values. In some embodiments of each or any of the above- or below-mentioned embodiments, the composition provides a reduction in the depression volume and area of skin contours in the region of acne scarring by greater than about 90%.

In some embodiments of each or any of the above- or below-mentioned embodiments, an individual may have almost complete removal of the acne scar by the treatments and methods described herein, wherein the acne depth, volume, and area are reduced by greater than about 90%.

In some embodiments of each or any of the above- or below-mentioned embodiments, the method improves skin color. In some embodiments, improving skin color comprises decreasing L* by about 1%, about 5%, about 10%, about 15%, about 20%, about 25% or greater than about 50%, or any amount defined by a range in between any two aforementioned values and/or increasing a* by about 1%, about 5%, about 10%, about 15%, about 20%, about 25% or greater than about 50%, or any amount defined by a range in between any two aforementioned values. In some embodiments of each or any of the above- or below-mentioned embodiments, L* is decreased by greater than about 50%. In some embodiments of each or any of the above- or below-mentioned embodiments, a* is increased by greater than about 50%. In some embodiments of each or any of the above- or below-mentioned embodiments, the composition provides an improvement in skin color.

In some embodiments of each or any of the above- or below-mentioned embodiments, the method and composition increases volume and area of elevations of skin contours in the region of acne scarring by about 10%, about 20%, about 30%, about 40%, about 50%, about 60%, about 70%, about 80% or greater than about 90% or any amount in between a range described by any two aforementioned values. In some embodiments of each or any of the above- or below-mentioned embodiments, volume and area of elevations of skin contours in the region of acne scarring is increased by greater than about 90%.

In some embodiments of each or any of the above- or below-mentioned embodiments, the method and composition levels the skin contours in a treatment area, wherein the treatment area comprises acne scarring or depressions.

In some embodiments of each or any of the above- or below-mentioned embodiments, the method and composition reduces the L* black to white scale in the treatment field as compared to a control treatment. In some embodiments of each or any of the above- or below-mentioned embodiments, the method and composition increases the a* green to red color scale in the composition treatment field as compared to a control treatment field.

The disclosure also provides methods to treat (e.g., restore) skin countours affected by acne scarring. The methods may comprise administering a composition that comprises tropoelastin to an area of skin with skin countours affected by acne scarring. In an embodiment, the methods comprise treating an area of skin with skin countours affected by acne scarring to reduce the acne scar depth, volume, and/or area.

### BRIEF DESCRIPTION OF THE DRAWINGS

Various features of illustrative embodiments of the present disclosure are described below with reference to the drawings. The illustrated embodiments are intended to illustrate, but not to limit, the present disclosure. The drawings contain the following figures.

FIG. 1 depicts a flow chart of a patient disposition.

FIGS. 2A to 2D show the clinical grading of acne scarring at screening and day 1. The grading is based on an acne scarring scale as described in Tan et al. 2010 (Journal of Cutaneous Medicine and Surgery, Vol 14, No 4 (July/August) (incorporated by reference herein). Scale: 0=no visible acne scars; 1=hardly visible acne scars from 2.5 m distance; 2=easily recognized acne scars, less than half the affected area involved; 3=easily recognized acne scars, more than half the affected area involved; 4=easily recognized acne scars, entire area involved; entire area involved with prominent atrophic scars. As shown, is the side with the TE implant side (30 mg/ml recombinant human tropoelastin crosslinked with 0.5% derivatized hyaluronic acid) during the screening visit (FIG. 2A) and the Saline Control side (Intradermal (i.d.) implants of isotonic saline) (FIG. 2B). As shown, is the side with the TE implant during the day 1 (FIG. 2C) and the Saline Control side during day 1 (FIG. 2D).

FIG. 3 shows blind third party review (BTPR) assessment of scars using Global Impression of Change scale (PAS1). Scale: −3=much worse; −2=moderately worse; −1=slightly worse; 0=no change; 1=slightly better, 2=moderately better; 3=much better.

FIG. 4 shows the correlation between screening and baseline visits.

FIGS. 5A and 5B show results for all camera settings for area and volume, the results from the camera are increasing with increasing severity score and hence suggests the camera is measuring a similar construct.

FIGS. 6A and 6B show the relationship between GIC (BTPR) and results for different camera settings.

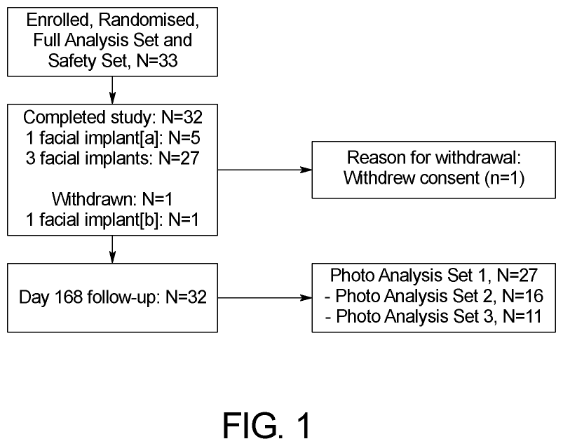

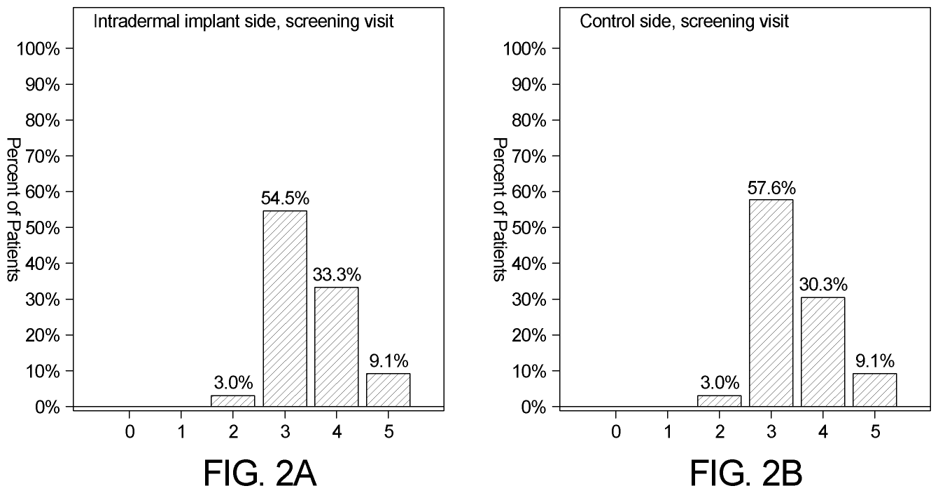

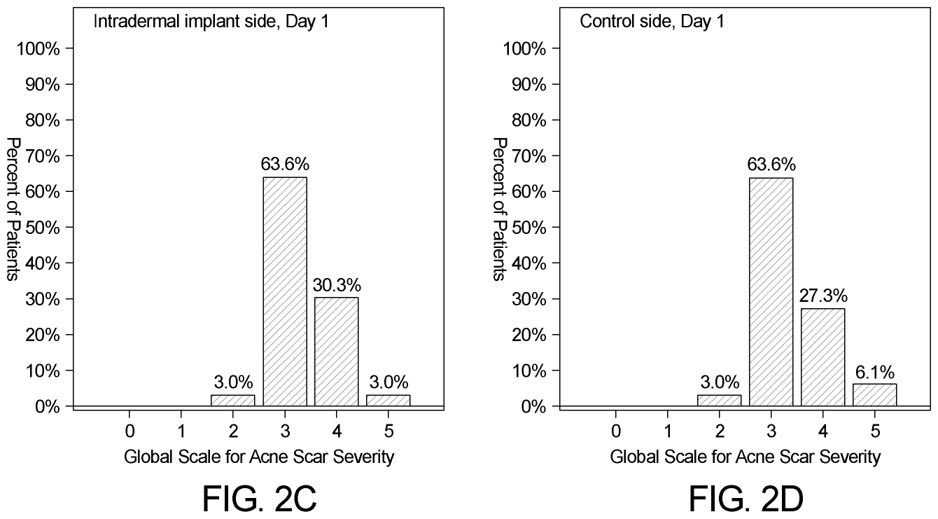

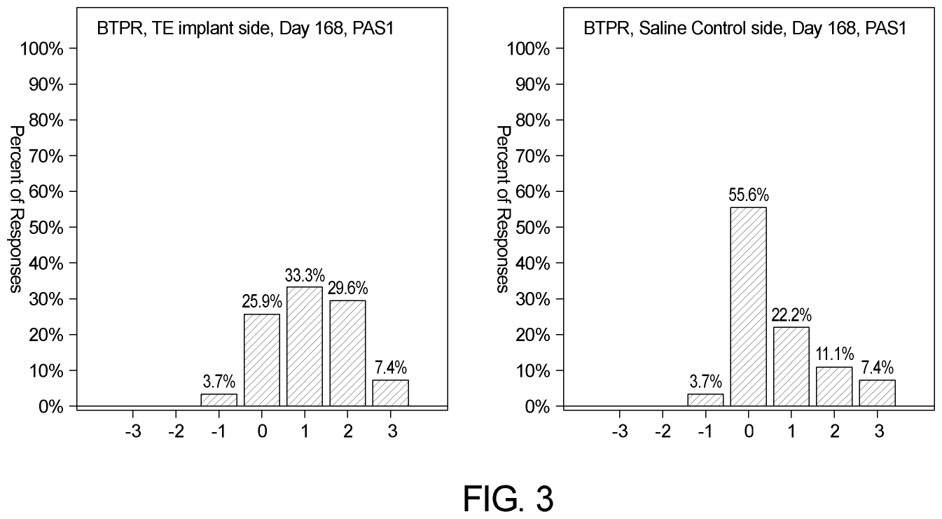

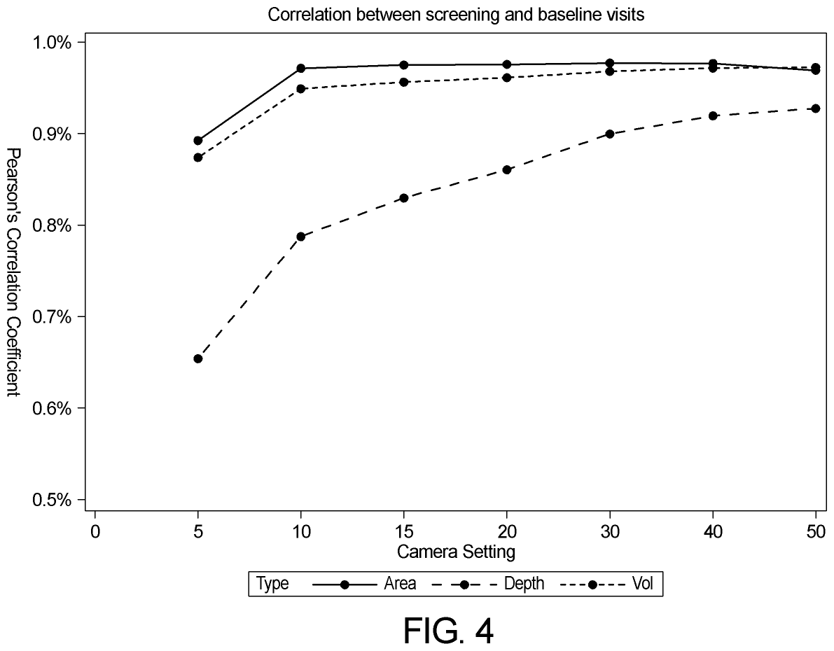

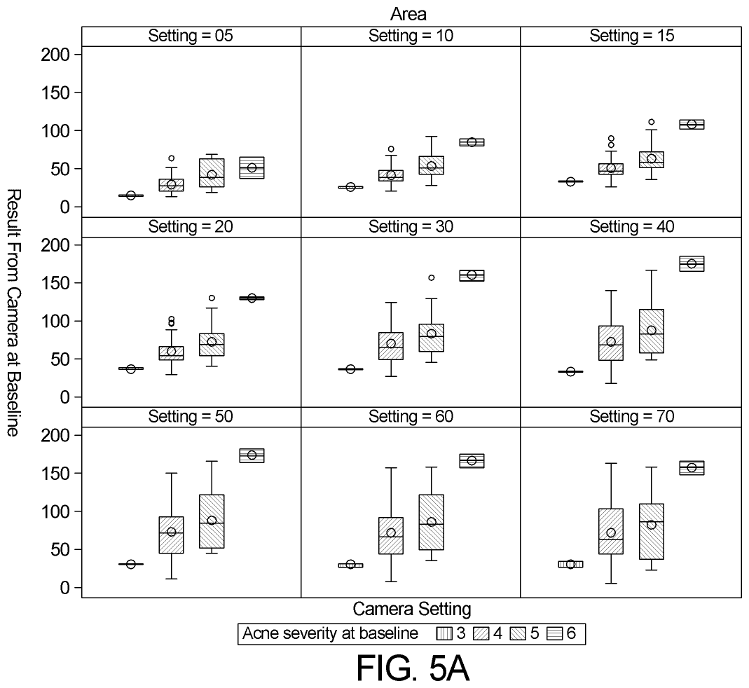

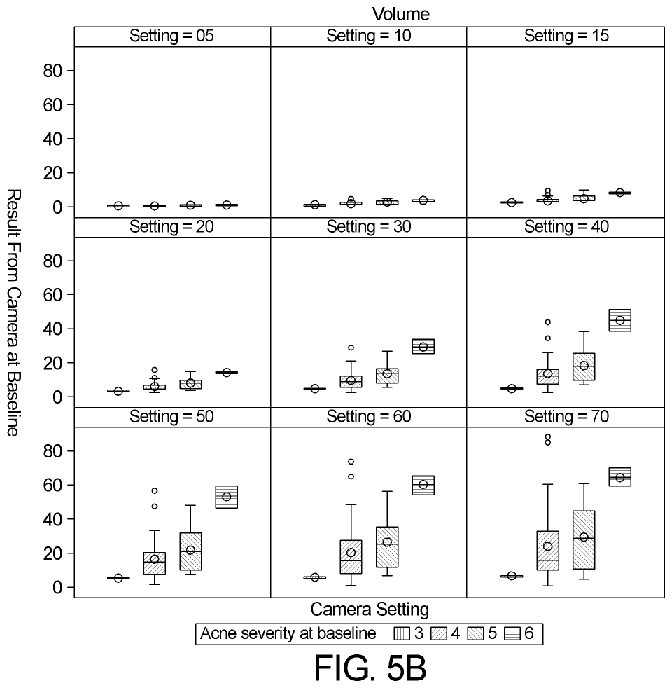

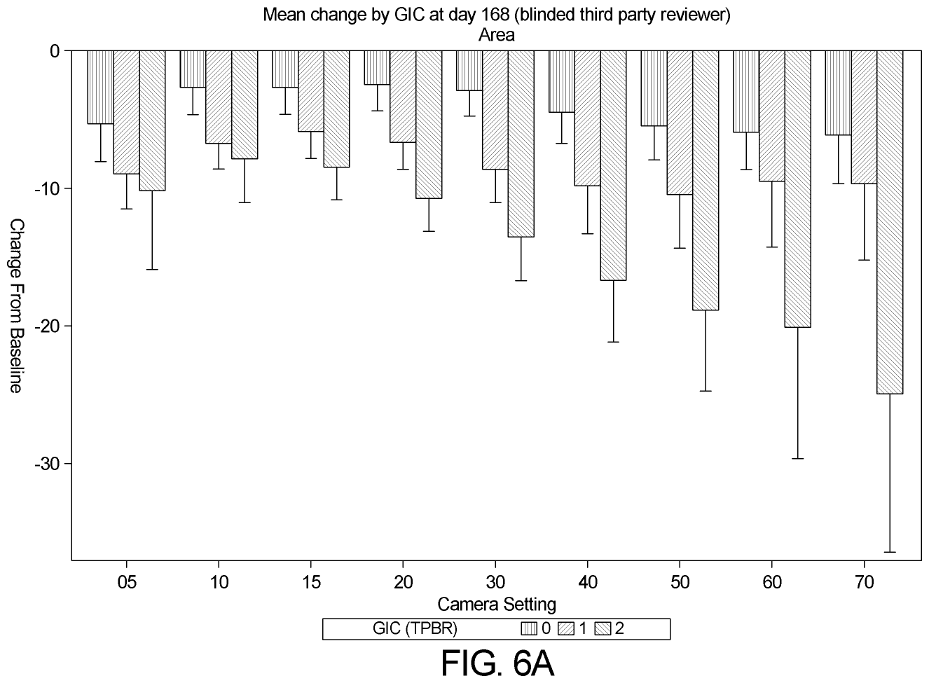

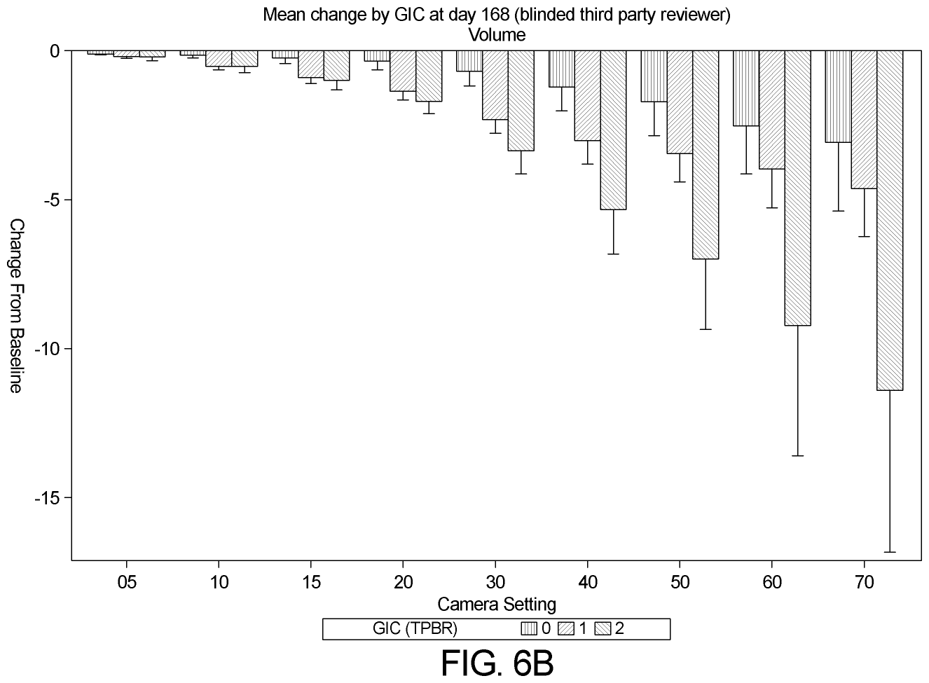

### DETAILED DESCRIPTION

Acne is a chronic inflammatory disease that may result from bacterial colonization of pores and hair follicles on the skin of the face, neck, chest and back. The bacterial colonization may be caused by bacteria, such as Propionibacterium acnes, for example. Acne may also be caused by increased sebum production, altered keratinization and inflammation. However, sometimes, the triggering of an acne flareup is unclear.

Acne may start at early puberty and into adulthood, during a time where there is increased oil production which may support the growth and colonization of several types of bacteria. Increased oil production may also be caused by hormones, stress and even diet.

Problems associated with acne include soreness, itchiness, pain and may also affect the quality of life. Although there are available treatments for preventing and healing acne, people may be left with the aftermath of their acne, such as unsightly and permanent acne scarring, which can also be highly visible. As acne affects an age group from pre-teens to adults, acne scarring affects pre-teens as well as adults, and can lead to loss of confidence and may lead to low self-esteem.

There are various degrees of scarring that can occur as a result of acne. Scarring may include rolling atrophic, ice-pick scars, boxcar scars and other types of deep depressions in this skin. The depth and extent of acne scarring may vary. Previous treatments have been described such as subcision, punch excision, laser resurfacing, dermabrasion and chemical peels. In some cases, the scars can also lead to discoloration of the skin.

Disclosed herein are methods of treating acne scarring which may help to decrease skin depression, decrease discoloration of the skin and/or even out the contours of the skin.

Unless defined otherwise, all technical and scientific terms used herein have the same meaning as commonly understood by one of ordinary skill in the art to which the invention pertains.

The terms “a,” “an,” “the” and similar referents used in the context of describing the invention (especially in the context of the following claims) are to be construed to cover both the singular and the plural, unless otherwise indicated herein or clearly contradicted by context.

“About” as used herein when referring to a measurable value is meant to encompass variations of +20% or +10% , more preferably +5% , even more preferably +1% , and still more preferably +0.1% from the specified value.

As used herein, except where the context requires otherwise, the term ‘comprise’ and variations of the term, such as “comprising,” “comprises” and “comprised,” are not intended to exclude further additives, components, integers or steps.

As used herein, “scarring,” “fibrosis,” or “fibrotic response” may refer to formation of fibrous (scar) tissue in response to injury or medical intervention.

A “scar,” as used herein, may refer to a mark left on the skin or within body tissue where a wound, such as a blemish or acne has healed and fibrous connective tissue has developed. As described herein are scars that may be a result from acne.

The term “tropoelastin” refers to a protein from which elastin is formed. Tropoelastin may be monomeric. Tropoelastin is generally not cross-linked, covalently or otherwise. Tropoelastin may reversibly coacervate. Thus, tropoelastin is distinguished from elastin because elastin consists of covalently cross linked tropoelastin which cannot reversibly coacervate. The tropoelastin may be human tropoelastin. Tropoelastin may be synthetic, for example it may be derived from recombinant expression or other synthesis, or it may be obtained from a natural source such as porcine aorta. As generally known in the art, tropoelastin may exist in the form of a variety of fragments. In some embodiments of each or any of the above- or below-mentioned embodiments, the composition provided in the methods herein comprises monomeric tropoelastin. In some embodiments of each or any of the above- or below-mentioned embodiments, the monomeric tropoelastin is cross-linked to HA. In some embodiments of each or any of the above- or below-mentioned embodiments, the tropoelastin comprises the sequence set forth in any one of SEQ ID NOs: 1-15.

In some embodiments of each or any of the above- or below-mentioned embodiments, the methods of the invention utilize the SHELδ26A tropoelastin analogue (WO 1999/03886) for the various applications described herein including for the compositions that are used in the described methods. The amino acid sequence of SHELδ26A is:

(SEQ ID NO: 1)
GGVPGAIPGGVPGGVFYPGAGLGALGGGALGPGGKPLKPVPGGLAGAGLGA
GLGAFPAVTFPGALVPGGVADAAAAYKAAKAGAGLGGVPGVGGLGVSAGAV
VPQPGAGVKPGKVPGVGLPGVYPGGVLPGARFPGVGVLPGVPTGAGVKPKA
PGVGGAFAGIPGVGPFGGPQPGVPLGYPIKAPKLPGGYGLPYTTGKLPYGY
GPGGVAGAAGKAGYPTGTGVGPQAAAAAAAKAAAKFGAGAAGVLPGVGGAG
VPGVPGAIPGIGGIAGVGTPAAAAAAAAAAKAAKYGAAAGLVPGGPGFGPG
VVGVPGAGVPGVGVPGAGIPVVPGAGIPGAAVPGVVSPEAAAKAAAKAAKY
GARPGVGVGGIPTYGVGAGGFPGFGVGVGGIPGVAGVPSVGGVPGVGGVPG
VGISPEAQAAAAAKAAKYGVGTPAAAAAKAAAKAAQFGLVPGVGVAPGVGV
APGVGVAPGVGLAPGVGVAPGVGVAPGVGVAPGIGPGGVAAAAKSAAKVAA
KAQLRAAAGLGAGIPGLGVGVGVPGLGVGAGVPGLGVGAGVPGFGAVPGAL
AAAKAAKYGAAVPGVLGGLGALGGVGIPGGVVGAGPAAAAAAAKAAAKAAQ
FGLVGAAGLGGLGVGGLGVPGVGGLGGIPPAAAAKAAKYGAAGLGGVLGGA
GQFPLGGVAARPGFGLSPIFPGGACLGKACGRKRK.

In some embodiments of each or any of the above- or below-mentioned embodiments, the tropoelastin isoform is the SHEL isoform (WO 1994/14958; included by reference in its entirety herein):

(SEQ ID NO: 2)
SMGGVPGAIPGGVPGGVFYPGAGLGALGGGALGPGGKPLKPVPGGLAGAGL
GAGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVKP
KAPGVGGAFAGIPGVGPFGGPQPGVPLGYPIKAPKLPGGYGLPYTTGKLPY
GYGPGGVAGAAGKAGYPTGTGVGPQAAAAAAAKAAAKFGAGAAGVLPGVGG
AGVPGVPGAIPGIGGIAGVGTPAAAAAAAAAAKAAKYGAAAGLVPGGPGFG
PGVVGVPGAGVPGVGVPGAGIPVVPGAGIPGAAVPGVVSPEAAAKAAAKAA
KYGARPGVGVGGIPTYGVGAGGFPGFGVGVGGIPGVAGVPSVGGVPGVGGV
PGVGISPEAQAAAAAKAAKYGVGTPAAAAAKAAAKAAQFGLVPGVGVAPGV
GVAPGVGVAPGVGLAPGVGVAPGVGVAPGVGVAPGIGPGGVAAAAKSAAKV
AAKAQLRAAAGLGAGIPGLGVGVGVPGLGVGAGVPGLGVGAGVPGFGAGAD
EGVRRSLSPELREGDPSSSQHLPSTPSSPRVPGALAAAKAAKYGAAVPGVL
GGLGALGGVGIPGGVVGAGPAAAAAAAKAAAKAAQFGLVGAAGLGGLGVGG
LGVPGVGGLGGIPPAAAAKAAKYGAAGLGGVLGGAGQFPLGGVAARPGFGL
SPIFPGGACLGKACGRKRK

or a protease resistant derivative of the SHEL or SHELδ26A isoforms (WO 2000/04043; included by reference in its entirety herein). As described in WO 2000/04043, the protein sequences of tropoelastin described may have a mutated sequence that leads to a reduced or eliminated susceptibility to digestion by proteolysis. Without being limiting, the tropoelastin amino acid sequence has a reduced or eliminated susceptibility to serine proteases, thrombin, kallikrein, metalloproteases, gelatinase A, gelatinase B, serum proteins, trypsin or elastase, for example In some embodiments of each or any of the above- or below-mentioned embodiments, the tropoelastin comprises a sequence set forth in SEQ ID NO: 3 (SHELδ26A isoform):

(SEQ ID NO: 3)
GGVPGAIPGGVPGGVFYPGAGLGALGGGALGPGGKPLKPVPGGLAGAGLGA
GLGAFPAVTFPGALVPGGVADAAAAYKAAKAGAGLGGVPGVGGLGVSAGAV
VPQPGAGVKPGKVPGVGLPGVYPGGVLPGARFPGVGVLPGVPTGAGVKPKA
PGVGGAFAGIPGVGPFGGPQPGVPLGYPIKAPKLPGGYGLPYTTGKLPYGY
GPGGVAGAAGKAGYPTGTGVGPQAAAAAAAKAAAKFGAGAAGVLPGVGGAG
VPGVPGAIPGIGGIAGVGTPAAAAAAAAAAKAAKYGAAAGLVPGGPGFGPG
VVGVPGAGVPGVGVPGAGIPVVPGAGIPGAAVPGVVSPEAAAKAAAKAAKY
GARPGVGVGGIPTYGVGAGGFPGFGVGVGGIPGVAGVPSVGGVPGVGGVPG
VGISPEAQAAAAAKAAKYGVGTPAAAAAKAAAKAAQFGLVPGVGVAPGVGV
APGVGVAPGVGLAPGVGVAPGVGVAPGVGVAPGIGPGGVAAAAKSAAKVAA
KAQLRAAAGLGAGIPGLGVGVGVPGLGVGAGVPGLGVGAGVPGFGAVPGAL
AAAKAAKYGAAVPGVLGGLGALGGVGIPGGVVGAGPAAAAAAAKAAAKAAQ
FGLVGAAGLGGLGVGGLGVPGVGGLGGIPPAAAAKAAKYGAAGLGGVLGGA
GQFPLGGVAARPGFGLSPIFPGGACLGKACGRKRK.

In some embodiments, the tropoelastin comprises a sequence set forth below (SHELδmod isoform):

(SEQ ID NO: 4)
GGVPGAVPGGVPGGVFYPGAGFGAVPGGVADAAAAYKAAKAGAGLGGVPGV
GGLGVSAGAVVPQPGAGVKPGKVPGVGLPGVYPGFGAVPGARFPGVGVLPG
VPTGAGVKPKAPGVGGAFAGIPGVGPFGGPQPGVPLGYPIKAPKLPGGYGL
PYTTGKLPYGYGPGGVAGAAGKAGYPTGTGVGPQAAAAAAAKAAAKFGAGA
AGFGAVPGVGGAGVPGVPGAIPGIGGIAGVGTPAAAAAAAAAAKAAKYGAA
AGLVPGGPGFGPGVVGVPGFGAVPGVGVPGAGIPVVPGAGIPGAAGFGAVS
PEAAAKAAAKAAKYGARPGVGVGGIPTYGVGAGGFPGFGVGVGGIPGVAGV
PSVGGVPGVGGVPGVGISPEAQAAAAAKAAKYGVGTPAAAAAKAAAKAAQF
GLVPGVGVAPGVGVAPGVGVAPGVGLAPGVGVAPGVGVAPGVGVAPGIGPG
GVAAAAKSAAKVAAKAQLRAAAGLGAGIPGLGVGVGVPGLGVGAGVPGLGV
GAGVPGFGAVPGALAAAKAAKYGAVPGVLGGLGALGGVGIPGGVVGAGPAA
AAAAAKAAAKAAQFGLVGAAGLGGLGVGGLGVPGVGGLGGIPPAAAAKAAK
YGAAGLGGVLGGAGQFPLGGVAARPGFGLSPIFPGGACLGKACGRKRK.

Tropoelastin analogues generally have a sequence that is homologous to a human tropoelastin sequence. Percentage identity between a pair of sequences may be calculated by the algorithm implemented in the BESTFIT computer program. Another algorithm that calculates sequence divergence has been adapted for rapid database searching and implemented in the BLAST computer program. In comparison to the human sequence, the tropoelastin polypeptide sequence may be about 60% identical at the amino acid level, 70% or more identical at the amino acid level, 80% or more identical at the amino acid level, 90% or more identical at the amino acid level, 95% or more identical at the amino acid level, 97% or more identical at the amino acid level, or greater than 99% identical at the amino acid level.

Conservative amino acid substitutions (e.g., Glu/Asp, Val/lle, Ser/Thr, Arg/Lys, Gln/Asn) may also be considered when making comparisons because the chemical similarity of these pairs of amino acid residues are expected to result in functional equivalency in many cases. Amino acid substitutions that are expected to conserve the biological function of the polypeptide would conserve chemical attributes of the substituted amino acid residues such as hydrophobicity, hydrophilicity, side-chain charge, or size.

Recombinant forms of tropoelastin can be produced as shown in WO 1999/03886. These sequences include:

(SEQ ID NO: 5)
SMGGVPGAIPGGVPGGVFYPGAGLGALGGGALGPGGKPLKPVPGGLAGAGL
GAGLGAFPAVTFPGALVPGGVADAAAAYKAAKAGAGLGGVPGVGGLGVSAG
AVVPQPGAGVKPGKVPGVGLPGVYPGGVLPGARFPGVGVLPGVPTGAGVKP
KAPGVGGAFAGIPGVGPFGGPQPGVPLGYPIKAPKLPGGYGLPYTTGKLPY
GYGPGGVAGAAGKAGYPTGTGVGPQAAAAAAAKAAAKFGAGAAGVLPGVGG
AGVPGVPGAIPGIGGIAGVGTPAAAAAAAAAAKAAKYGAAAGLVPGGPGFG
PGVVGVPGAGVPGVGVPGAGIPVVPGAGIPGAAVPGVVSPEAAAKAAAKAA
KYGARPGVGVGGIPTYGVGAGGFPGFGVGVGGIPGVAGVPSVGGVPGVGGV
PGVGISPEAQAAAAAKAAKYGVGTPAAAAAKAAAKAAQFGLVPGVGVAPGV
GVAPGVGVAPGVGLAPGVGVAPGVGVAPGVGVAPGIGPGGVAAAAKSAAKV
AAKAQLRAAAGLGAGIPGLGVGVGVPGLGVGAGVPGLGVGAGVPGFGAGAD
EGVRRSLSPELREGDPSSSQHLPSTPSSPRVPGALAAAKAAKYGAAVPGVL
GGLGALGVGIPGGVVGAGPAAAAAAAKAAAKAAQFGLVGAAGLGGLGVGGL
GVPGVGGLGGIPPAAAAKAAKYGAAGLGGVLGGAGQFPLGGVAARPGFGLS
PIFPGGACLGKACGRKRK;:
(SEQ ID NO: 6)
GGVPGAIPGGVPGGVFYPGAGLGALGGGALGPGGKPLKPVPGGLAGAGLGA
GLGAFPAVTFPGALVPGGVADAAAAYKAAKAGAGLGGVPGVGGLGVSAGAV
VPQPGAGVKPGKVPGVGLPGVYPGGVLPGARFPGVGVLPGVPTGAGVKPKA
PGVGGAFAGIPGVGPFGGPQPGVPLGYPIKAPKLPGGYGLPYTTGKLPYGY
GPGGVAGAAGKAGYPTGTGVGPQAAAAAAAKAAAKFGAGAAGVLPGVGGAG
VPGVPGAIPGIGGIAGVGTPAAAAAAAAAAKAAKYGAAAGLVPGGPGFGPG
VVGVPGAGVPGVGVPGAGIPVVPGAGIPGAAVPGVVSPEAAAKAAAKAAKY
GARPGVGVGGIPTYGVGAGGFPGFGVGVGGIPGVAGVPSVGGVPGVGGVPG
VGISPEAQAAAAAKAAKYGVGTPAAAAAKAAAKAAQFGLVPGVGVAPGVGV
APGVGVAPGVGLAPGVGVAPGVGVAPGVGVAPGIGPGGVAAAAKSAAKVAA
KAQLRAAAGLGAGIPGLGVGVGVPGLGVGAGVPGLGVGAGVPGFGAVPGAL
AAAKAAKYGAAVPGVLGGLGALGGVGIPGGVVGAGPAAAAAAAKAAAKAAQ
FGLVGAAGLGGLGVGGLGVPGVGGLGGIPPAAAAKAAKYGAAGLGGVLGGA
GQFPLGGVAARPGFGLSPIFPGGACLGKACGRKRK;
(SEQ ID NO: 7)
MGGVPGAVPGGVPGGVFYPGAGFGAVPGGVADAAAAYKAAKAGAGLGGVPG
VGGLGVSAGAVVPQPGAGVKPGKVPGVGLPGVYPGFGAVPGARFPGVGVLP
GVPTGAGVKPKAPGVGGAFAGIPGVGPFGGPQPGVPLGYPIKAPKLPGGYG
LPYTTGKLPYGYGPGGVAAAGKAGYPTGTGVGPQAAAAAAAKAAAKFGAGA
AGFGAVPGVGGAGVPGVPGAIPGIGGIAGVGTPAAAAAAAAAAKAAKYGAA
AGLVPGGPGFGPGVVGVPGFGAVPGVGVPGAGIPVVPGAGIPGAAGFGAVS
PEAAAKAAAKAAKYGARPGVGVGGIPTYGVGAGFFPGFGVGVGGIPGVAGV
PSVGGVPGVGGVPGVGISPEAQAAAAAKAAKYGVGTPAAAAAKAAAKAAQF
GLVPGVGVAPGVGVAPGVGVAPGVGLAPGVGVAPGVGVAPGVGVAPGIGPG
GVAAAAKSAAKVAAKAQLRAAAGLGAGIPGLGVGVGVPGLGVGAGVPGLGV
GAGVPGFGAVPGALAAAKAAKYGAVPGVLGGLGALGGVGIPGGVVGAGPAA
AAAAAKAAAKAAQFGLVGAAGLGGLGVGGLGVPGVGGLGGIPPAAAAKAAK
YGAAGLGGVLGGAGQFPLGGVAARPGFGLSPIFPGGACLGKACGRKRK;
(SEQ ID NO: 8)
SAMGGVPGALAAAKAAKYGAAVPGVLGGLGALGGVGIPGGVVGAGPAAAAA
AAKAAAKAAQFGLVGAAGLGGLGVGGLGVPGVGGLGGIPPAAAAKAAKYGA
AGLGGVLGGAGQFPLGGVAARPGFGLSPIFPGGACLGKACGRKRK;
(SEQ ID NO: 9)
SAMGALVGLGVPGLGVGAGVPGFGAGADEGVRRSLSPELREGDPSSSQHLP
STPSSPRVPGALAAAKAAKYGAAVPGVLGGLGALGGVGIPGGVVGAGPAAA
AAAAKAAAKAAQFGLVGAAGLGGLGVGGLGVPGVGGLGGIPPAAAAKAAKY
GAAGLGGVLGGAGQFPLGGVAARPGFGLSPIFPGGACLGKACGRKRK;
(SEQ ID NO: 10)
GIPPAAAAKAAKYGAAGLGGVLGGAGQFPLGGVAARPGFGLSPIFPGGACL
GKACGRKRK;
(SEQ ID NO: 11)
GAAGLGGVLGGAGQFPLGGVAARPGFGLSPIFPGGACLGKACGRKRK;
(SEQ ID NO: 12)
GADEGVRRSLSPELREGDPSSSQHLPSTPSSPRV;
(SEQ ID NO: 13)
GADEGVRRSLSPELREGDPSSSQHLPSTPSSPRF;
(SEQ ID NO: 14)
AAAGLGAGIPGLGVGVGVPGLGVGAGVPGLGVGAGVPGFGAGADEGVRRSL
SPELREGDPSSSQHLPSTPSSPRVPGALAAAKAAKYGAAVPGVLGGLGALG
GVGIPGGVVGAGPAAAAAAAKAAAKAAQFGLVGAAGLGGLGVGGLGVPGVG
GLGGIPPAAAAKAAKYGAAGLGGVLGGAGQFPLGGVAARPGFGLSPIFPGG
ACLGKACGRKRK;
and
(SEQ ID NO: 15)
AAAGLGAGIPGLGVGVGVPGLGVGAGVPGLGVGAGVPGFGAVPGALAAAKA
AKYGAAVPGVLGGLGALGGVGIPGGVVGAGPAAAAAAAKAAAKAAQFGLVG
AAGLGGLGVGGLGVPGVGGLGGIPPAAAAKAAKYGAAGLGGVLGGAGQFPL
GGVAARPGFGLSPIFPGGACLGKACGRKRK.

Tropoelastin may be utilized in a form in which it is linked (e.g., covalently linked) to another molecule such as a biopolymer such as hyaluronic acid. In some embodiments, the tropoelastin is cross-linked to hyaluronic acid. In some embodiments, the composition comprises monomeric tropoelastin. In some embodiments of each or any of the above- or below-mentioned embodiments, the tropoelastin comprises the amino acid sequence set forth in any one of SEQ ID Nos: 1-15.

It is particularly preferred that where tropoelastin is linked to another molecule, the linkage does not impede or limit the biological properties of an unlinked form of tropoelastin.

The purpose of linking tropoelastin with another molecule is typically to enable tropoelastin to be localized to a region and to minimize the likelihood of the tropoelastin diffusing or otherwise migrating from that region.

In some embodiments of each or any of the above- or below-mentioned embodiments, the tropoelastin has a specified degree of purity with respect to the amount of tropoelastin in the composition, as compared with amounts of other proteins or molecules in the composition. In one embodiment, the tropoelastin is in a composition that has at least 75% purity, preferably 85% purity, more preferably more than 90% or 95% purity. Fragments of tropoelastin, i.e., truncated forms of a tropoelastin isoform that arise unintentionally through tropoelastin manufacture may be regarded as an impurity in this context.

It will further be understood that in certain embodiments the tropoelastin may be provided in the form of a composition that consists of or consists essentially of tropoelastin, preferably a full-length isoform of tropoelastin. In some embodiments of each or any of the above- or below-mentioned embodiments, the tropoelastin will be at least about 65% of the length of the relevant tropoelastin isoform, more than about 80% of the full length, more than about 90% or more than about 95% of the full length.

The term “hyaluronic acid” or “HA” may include hyaluronic acid and any of its hyaluronate salts, including, for example, sodium hyaluronate (the sodium salt), potassium hyaluronate, magnesium hyaluronate, and calcium hyaluronate. Hyaluronic acid from a variety of sources may be used herein. For example, hyaluronic acid may be extracted from animal tissues, harvested as a product of bacterial fermentation, or produced in commercial quantities by bioprocess technology. In some embodiments, the composition provided in the methods herein comprises monomeric tropoelastin. In some embodiments, the monomeric tropoelastin is cross-linked to HA. In some embodiments of each or any of the above- or below-mentioned embodiments, the tropoelastin comprises the sequence set forth in any one of SEQ ID NOs: 1-15. In some embodiments of each or any of the above- or below-mentioned embodiments, tropoelastin comprises the sequence set forth in SEQ ID NO: 1.

In some embodiments of each or any of the above- or below-mentioned embodiments, the composition comprises between 1-100 mg/ml tropoelastin crosslinked with about 0.1% to about 10% derivatized hyaluronic acid (HA). In some embodiments of each or any of the above- or below-mentioned embodiments, the composition comprises between 1-100 mg/ml tropoelastin crosslinked with about 0.4% to about 1% derivatized hyaluronic acid (HA). In some embodiments of each or any of the above- or below-mentioned embodiments, the tropoelastin is crosslinked with about 0.4%, about 0.5%, about 0.6%, about 0.8%, about 0.9% or about 1% derivatized hyaluronic acid (HA). In some embodiments, the composition comprises 30 mg/ml recombinant human tropoelastin crosslinked with 0.5% derivatized hyaluronic acid. In some embodiments of each or any of the above- or below-mentioned embodiments, the composition further comprises a buffer, wherein the buffer is phosphate buffered saline. In some embodiments, the composition comprises derivatized HA or underivatized HA, to control the extent to which the HA crosslinks with itself and/or the monomeric protein. In some embodiments of each or any of the above- or below-mentioned embodiments, the derivatized HA cross-links with the TE. In some embodiments, the tropoelastin is monomeric. In some embodiments of each or any of the above- or below-mentioned embodiments, the monomeric tropoelastin is released from the composition.

In some embodiments of each or any of the above- or below-mentioned embodiments, the HA may comprise, at least one linkable moiety, such as at least one cross-linkable moiety, for example, a carboxyl group, a hydroxyl group, an amine, a thiol, an alcohol, an alkene, an alkyne, a cyano group, or an azide, and/or modifications, derivatives, or combinations thereof

In some embodiments of each or any of the above- or below-mentioned embodiments, the HA may comprise, a spacer group, such that the spacer group can link to the same and/or a second molecule, for example, a second biomolecule or biopolymer.

The HA may be in the range of about 25 to about 10000 disaccharide units or residues. In some embodiments of each or any of the above- or below-mentioned embodiments, hyaluronic acid may be used in the range of 25 to 7,500 disaccharide units or residues.

In some embodiments of each or any of the above- or below-mentioned embodiments, the HA may be low or high molecular weight HA. High molecular weight HA (also referred to as “HMW HA”) as described herein generally describes a hyaluronic acid having a molecular weight of at least about 1.0 million Daltons (mw≥10⁶ or 1 MDa) to about 4.0 MDa. Low molecular weight HA (sometimes herein referred to as “LMW HA”) as used herein, generally describes a hyaluronic acid having a molecular weight of less than about 1.0 MDa.

In some embodiments of each or any of the above- or below-mentioned embodiments, the HA may be activated and/or modified with an activating agent, such as EDC or allylglycidyl ether, and/or modifying agent, such as NHS, HOBt or Bromine.

The term “Fitzpatrick scale” is given its plain and ordinary meaning, in view of this paper and without limitation, may refer to a scientific skin type classification. The Fitzpatrick scale has several different types from types 1-6. This scale is used by dermatologists and aesthetic medicine practitioners to determine which treatments are best suited for different skin types. The scale was developed to measure how skin reacts to ultraviolet light such as sun exposure. Additionally, this knowledge can be used by aesthetic doctors and laser technicians to know before administering a laser or another type of treatment. Type 1—typically has light, ivory skin but when exposed to the sun always burns and peels but never tans. Type 2—has a light, fair complexion and burns quickly when exposed to the sun and rarely tans. Type 3—usually has a beige tint to the skin and may burn when exposed to the sun but is capable of tanning. Type 4—has an olive skin or light brown tone and will not freckle when exposed to the sun. This person rarely gets a sun burn and tans regularly. Type 5—has a dark brown or black skin tone, rarely gets a sun burn and always tans under sun exposure. Type 6—has black and is the darkest skin tone. This person never burns and tans quickly when exposed to the sun. This helpful scale may be used by dermatologists to gauge certain skin's reaction to the sun and how to more quickly identify potentially malignant sun spots. Aesthetic doctors may also use the scale to determine the effectiveness of treatments on different skin types. In some embodiments of each or any of the above- or below-mentioned embodiments, the methods for treating acne scars may be used for individuals that may have the skin type classification of any one of types 1-6 on the Fitzpatrick scale.

The term “intradermal implant” is an implant that may go underneath the epidermis. Without being limiting, this may be in the area of skin such as the dermis, subdermis or hypodermis layer, for example. In some embodiments of each or any of the above- or below-mentioned embodiments, an implant may be placed intradermally.

In some embodiments of each or any of the above- or below-mentioned embodiments, an implant may be placed within the subcutaneous layer. This placement may be in addition to an intradermal placement of an implant.

A “retrograde linear threading technique” is a method for placing threads of implant into a target tissue. Firstly, the needle is inserted. Secondly, the implant is placed by depressing the plunger of the syringe at the same time as the needle is being withdrawn.

“Cross-hatching” is a method of placing threads of implant in a pattern that creates a “mat” within the target tissue. The movement of the needle in the tissue to complete linear threading in a crosshatching arrangement will disrupt fibrous tissue that is in the way or the path of the needle.

“Acne” as described herein, refers to a skin disease that occurs when hair follicles are clogged with dead skin cells and oil from the skin. It may be characterized by blackheads or whiteheads, pimples and oily skin. In some cases, acne may lead to scarring. In some embodiments of each or any of the above- or below-mentioned embodiments, the patient in need has acne as well as acne scars.

“Acne scars” as described herein, may be caused by inflammation within the skin. The acne scars may be due to abnormal healing following the inflammation caused by the acne. “Atrophic acne scars” may be caused by the loss of collagen from the healing response and are the most common cause of acne scarring.

“Ice pick scars,” caused by acne may be narrow deep scars that extend into the dermis. Ice pick scar is a type of atrophic acne scar.

“Rolling atrophic acne scar” is an indented scar that heals below the normal layer of skin tissue.

“Boxcar scar” is a type of acne scarring that may appear as indentations into the skin. They are defined by their sharp edges that may move straight down into the skin and are usually a different size from the acne lesion from which they originated. Boxcar scar is a type of atrophic acne scar.

“Hyperpigmentation” is the darkening of skin caused by increase in melanin. Without being limiting, causes of hyperpigmentation include sun damage, inflammation, skin injury and acne. The skin may respond to damage by the excess production of melanin that is produced from melanocytes. In some embodiments of each or any of the above- or below-mentioned embodiments, the scar comprises a silver coloring prior to the administration of the composition. In some embodiments of any one of each or any of the above- or below-mentioned embodiments, administering the composition decreases the silver coloring and increases the dark red or pink coloring of the scar. In some embodiments of any one of each or any of the above- or below-mentioned embodiments, the method decreases L* by 1%, 5%, 10%, 15%, 20%, 25% or greater than 50%, or any amount defined by a range in between any two aforementioned values and increases a* by 1%, 5%, 10%, 15%, 20%, 25% or greater than 50%, or any amount defined by a range in between any two aforementioned values. The methods described herein may also be used to treat acne scarring and to decrease the discoloration of the skin that has been caused by the acne.

The “CIELAB color space” is used to define the skin color and color change that may occur following treatment of the skin with the composition comprising tropoelastin. The CIELAB color space (also known as CIE L*a*b* or sometimes abbreviated as simply “Lab” color space) is a color space defined by the International Commission on Illumination (CIE) in 1976. It expresses color as three values: L* for the lightness from black (0) to white (100), a* from green (−) to red (+), and b* from blue (−) to yellow (+).

### Methods of Treating a Scar

Methods are provided herein for treating an acne scar (e.g., a fibrotic acne scar) in a patent in need thereof including, for example, improving the color and/or appearance of an acne scar. The methods may comprise administering a composition that comprises tropoelastin to an area of skin in the patient having a scar including, for example, administering the composition adjacent to or directly into or beneath the scar. In another embodiment, the composition comprising tropoelastin is also placed around the scar, such as beneath the scar and around edges of the scar. The depth, affected area and/or volume of the acne scar is reduced following administering of the composition (including as compared to an otherwise identical composition lacking tropoelastin such as a saline control). Additonally or alternatively, the disclosed methods may increase the elevation volume and area of elevations of skin contours in the region of acne scarring (including as compared to an otherwise identical composition lacking tropoelastin such as a saline control).

The methods may further comprise needle disruption of fibrotic elements in the acne scar, such as fibrotic strands or elements (e.g., fibrotic tissue) underneath the scar to cut or release the fibrotic strands that are tethering the surface of the skin deep into a depression. In an embodiment, disruption of the fibrotic elements creates a dermal pocket beneath the acne scar and the composition is administered into the dermal pocket. After disruption of the scar tissue, the dermal pocket may then be filled with the disclosed compositions.

The disruption of a scar may be performed with a 18G-32G needle, such as a 18G, 21G, 23G, 25G, 27G, 29G or 30G needle. Without being limiting, disruption of the fibrotic elements may be performed for scars, such as rolling atrophic, boxcar or icepick scars. The administering comprises injecting the composition into the dermal pocket. The composition is administered as an injection beneath the acne scar. The composition comprising tropoelastin is also placed around the scar, such as beneath the scar and/or around the edges of the scar. An even or uniform placement of the composition comprising tropoelastin around the scar throughout the dermis surrounding the scar may ensure that the tropoelastin product is around the scar area and may enable a more effective treatment. The composition is administered in a volume of about 10 μL to about 100 μL per implant/injection. The injection is given using a retrograde linear threading technique in a cross-hatching arrangement to ensure any fibrous strands within the acne scar are disrupted.

In some embodiments, disruption of fibrous strands are performed for atrophic scars.

Disrupting of the fibrotic strands may be performed using the needle to disrupt strands which may tether the skin to the underlying tissue and contribute to the depression to create a pocket to make space for the tropoelastin composition to be placed under the acne scar, wherein the fibrotic strands are located. Thus, the tropoelastin composition will be available to the skin tissue cells immediately around the scar so they can grow in and around the product which would remodel the fibrotic elements of the scar.

The methods disclosed herein may lead to the remodeling of the fibrotic elements surrounding the scar.

For basic or classic subcision the needle can be as large as 18G, usually 21G or 23G, for example, however a 27G or 30G needle may also be used as it is small enough to allow some subcision but also enables the creation of a pocket or space around the scar and in the dermal skin layer for placement of the product.

The needle is inserted at an angle of about 30° parallel to the skin, wherein the needle comprises a bevel and the bevel is facing upwards. The needle is inserted more than one time to break up the fibrous strands and create the dermal pocket. Even pressure is applied to inject the composition at the same time as the needle is withdrawn from the dermal pocket. The administering of the composition is repeated, wherein one or more bolus injections into the scar is administered.

The methods disclosed herein may advantageously reduce the depth, affected surface area, and/or volume of the acne scar. Indeed, small depressions, as well as large depressions, were shown to improve at 168 days after administration of the composition (e.g., 30 mg/ml tropoelastin cross-linked with 0.5% dHA) with increased smoothness and/or leveling of skin countours. Surprisingly, this also led to the repair of atrophic scars during the remodeling and maturation phase to reduce the scars appearance.

The methods disclosed herein may be used to improve the appearance of atrophic acne scars. As described herein, treatments may be administered at days 0, 28 and 56. Such treatments surprisingly led to improved appearance and coloration of the skin in several patients including, patients with rolling, boxcar or icepick type acne scarring. In some embodiments, wherein the coloration of the scar comprises a silver color, the methods disclosed herein decrease the silver coloring of the scar and/or increase the red coloring or pink coloring of the scar.

The compositions disclosed herein may comprise about 1 mg/ml to about 400 mg/ml tropoelastin (e.g., 1 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, 200 mg/ml, 210 mg/ml, 220 mg/ml, 230 mg/ml, 240 mg/ml, 250 mg/ml, 260 mg/ml, 270 mg/ml, 280 mg/ml, 290 mg/ml, 300 mg/ml, 310 mg/ml, 320 mg/ml, 330 mg/ml, 340 mg/ml, 350 mg/ml, 360 mg/ml, 370 mg/ml, 380 mg/ml, 390 mg/ml or 400 mg/ml tropoelastin).

The compositions disclosed herein may also comprise hyaluronic acid (HA). In a further embodiment, the tropoelastin is crosslinked with about 0.1% to about 10% derivatized hyaluronic acid. In yet a further embodiment, the composition comprises about 30 mg/ml human tropoelastin (e.g., recombinant human tropoelastin) crosslinked with about 0.5% derivatized hyaluronic acid. The composition may further comprise a buffer such as phosphate buffered saline.

Also provided herein are pharmaceutical formulations that comprise the disclosed compositions comprising tropoelastin.

A maximum volume of the composition disclosed herein that is administered is between about 100 μL to about 5 mL (e.g., between about 100 μL to about 500 μL per square cm).

In an embodiment, the patient in need thereof has a skin type on a Fitzpatrick skin type scale of I, II, III, IV, V, or VI.

The scar treated by the methods disclosed herein may comprise a grade of any one of 0, 1, 2, 3, 4 or 5 as described in a Global Scale for Acne Scar Severity (SCAR-S) by Tan et al. 2010 (Journal of Cutaneous Medicine and Surgery, Vol 14, No 4 (July/August), incorporated by reference herein). The grading is as follows: 0: Clear; No visible scars from acne, 1: Almost clear; Hardly visible scars from 2.5 m away, 2: Mild; Easily recognizable; less than half the affected area (e.g., face, back or chest) involved; 3: Moderate; More than half the affected area (e.g., face, back or chest) involved; 4: Severe; Entire area involved, 5: Very severe; Entire area with prominent atrophic or hypertrophic scars.

In an embodiment, the acne scar may be an ice pick scar, a boxcar scar, a rolling atrophic acne scar, a distensible rolling atrophic acne scar, or a hypertrophic scar. The scar may be on a subject's face, back or torso.

The acne scar treated by the methods disclosed herein may have a depth of about 0.1 mm to about 5 mm (e.g., about 0.1 mm, about 0.5 mm, about 1.0 mm, about 1.5 mm, about 2 mm, about 2.5 mm, about 3.0 mm, about 3.5 mm. about 4.0 mm, about 4.5 mm, about 5 mm, or any depth in a range in between any two aforementioned values).

The acne scar treated by the methods disclosed herein may have an affected area of about 0.05 mm² to about 400 mm².

The acne scar treated by the methods disclosed herein may have a volume of about 0.01 mm³ to 2,000 mm³ (e.g., about 0.01 mm³, about 1 mm³, about 10 mm³, about 20 mm³, about 30 mm³, about 40 mm³, about 50 mm³, about 60 mm³, about 70 mm³, about 80 mm³, about 90 mm³, about 100 mm³, about 125 mm³, about 150 mm³, about 175 mm³, about 200 mm³, about 225 mm³, about 250 mm³, about 275 mm³, about 300 mm³, about 325 mm³, about 350 mm³, about 375 mm³, about 400 mm³, about 425 mm³, about 450 mm³, about 475 mm³, about 500 mm³, about 525 mm³, about 550 mm³, about 575 mm³, about 600 mm³, about 625 mm³, about 650 mm³, about 675 mm³, about 700 mm³, about 725 mm³, about 750 mm³, about 775 v, about 800 mm³, about 825 mm³, about 850 mm³, about 875 mm³, about 900 mm³, about 925 mm³, about 950 mm³, 975 mm3, about 1,000 mm³, about 1,100 mm³, about 1,200 mm³, about 1,300 mm³, about 1,400 mm³, about 1,500 mm³, about 1,600 mm³, about 1,700 mm³, about 1,800 mm³, about 1,900 mm³, or about 2,000 mm³, or any volume in a range between any two aforementioned values).

The scar treated by the methods disclosed herein may decrease a skin color defined by CIE L*a*b* color coordinates (e.g., decreases L* by about 1%, about 5%, about 10%, about 15%, about 20%, about 25% or greater than about 50%, or any amount defined by a range in between any two aforementioned values). In a preferred embodiment, the methods disclosed herein decrease L* by greater than about 50%.

The methods disclosed herein may further increase a*, including by about 1%, about 5%, about 10%, about 15%, about 20%, about 25% or greater than about 50%, or any amount defined by a range in between any two aforementioned values. In a preferred embodiment, the methods disclosed herein increase a* by greater than about 50%.

In an embodiment, the scar comprises a color that is different from a natural skin coloring of the patient and the compositions disclosed herein results in the color of the scar decreasing or fading in color intensity such that the color of the scar integrates into the natural skin coloring of the patient.

The compositions disclosed herein may improve the appearance of a scar (e.g., reduce the depth, volume, surface area, and/or color of the scar including, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more as compared to an otherwise identical composition lacking tropoelastin or a saline solution) within about 1 month, about 2 months, about 3 months, about 4 months, about 5 months, about 6 months, about 7 months, about 8 months, about 9 months, about 10 months, about 11 months, or about 12 months after injection in an area of skin having the scar.

The following examples, sequence listing and figures are provided to aid the understanding of the present invention, the true scope of which is set forth in the appended claims. It is understood that modifications can be made in the procedures set forth without departing from the spirit of the invention.

1-33. (canceled)

34. A method of reducing the depth, affected area, and/or volume of an acne scar in a patient in need thereof, the method comprising administering a composition that comprises tropoelastin and hyaluronic acid to the area of skin having the acne scar,
wherein the composition comprises between about 1 mg/ml to about 250 mg/ml tropoelastin, and
wherein the tropoelastin is crosslinked with about 0.1% to about 10% hyaluronic acid.

35. The method of claim 34, wherein the composition comprises between about 1 to about 100 mg/ml tropoelastin crosslinked with about 0.4% to about 1% hyaluronic acid (HA).

36. The method of claim 34, wherein the hyaluronic acid is derivatized hyaluronic acid.

37. The method of claim 34, wherein the composition comprising tropoelastin is administered beneath the scar and/or around edges of the scar.

38. The method of claim 34, wherein the method further comprises a step of disrupting fibrotic strands underneath the acne scar.

39. The method of claim 38, wherein the disrupting step is performed prior to administration of the composition.

40. The method of claim 38, wherein the step of disrupting the fibrotic strands creates a dermal pocket underneath the acne scar.

41. The method of claim 40, wherein the administering step comprises injecting the composition into the dermal pocket.

42. The method of claim 34, wherein the administering step is by injection, and wherein the injection is administered using a retrograde linear threading technique in a cross-hatching arrangement.

43. The method of claim 34, wherein the administering step is repeated, and wherein one or more bolus injections are administered.

44. The method of claim 34, wherein the treatment reduces the acne scar affected area by at least about 10%.

45. The method of claim 34, wherein the treatment reduces the acne scar volume by at least about 10%.

46. The method of claim 34, wherein the treatment reduces the acne scar depth by at least about 10%.

47. The method of claim 34, wherein the treatment further improves skin color of the acne scar.

48. The method of claim 34, wherein the tropoelastin is recombinant human tropoelastin.

49. A method of restoring skin contours affected by acne scarring in a subject in need thereof, the method comprising: administering a composition that comprises tropoelastin to an area of skin having skin contours affected by acne scarring, wherein the composition comprises between about 1 mg/ml to about 250 mg/ml tropoelastin, and wherein the tropoelastin is crosslinked with about 0.1% to about 10% derivatized hyaluronic acid.

50. The method of claim 49, wherein the composition comprises between about 1 to about 100 mg/ml tropoelastin crosslinked with about 0.4% to about 1% derivatized hyaluronic acid (HA).

51. A composition for reducing the depth, affected area, and/or volume of an acne scar comprising between about 1 mg/ml to about 250 mg/ml tropoelastin crosslinked with about 0.1% to about 10% derivatized hyaluronic acid.

52. The composition of claim 51, wherein the composition comprises between about 1 to about 100 mg/ml tropoelastin crosslinked with about 0.4% to about 1% derivatized hyaluronic acid (HA).

53. The composition of claim 52, wherein the tropoelastin comprises about 30 mg/ml recombinant human tropoelastin crosslinked with about 0.5% derivatized hyaluronic acid.
